(19) United States
(12) Patent Application Publication
(10) Pub. No.: US 2007/0116747 A1
(43) Pub. Date: May 24, 2007

(54) Disposable Plastic Glove with Antiseptic Coating

(76) Inventor: Hew Seng Yeap, Rayong (TH)

Correspondence Address: WHYTE HIRSCHBOECK DUDEK S C, 555 EAST WELLS STREET, SUITE 1900, MILWAUKEE, WI, 53202, US

(21) Appl. No.: 11/539,003

(22) Filed: Oct. 5, 2006

Related U.S. Application Data
(60) Provisional application No. 60/724,175, filed on Oct. 6, 2005.

(51) Int. Cl. A61K 31/44; A61K 9/70

(52) U.S. Cl. 424/443; 514/358

(57) ABSTRACT

A disposable plastic glove is provided the inside surface of which carries an antiseptic agent. The glove is thin and elastic, and it can be made of any material that will produce a glove with such attributes. Preferred materials include both natural and synthetic rubber latex, acrylonitrile and polyvinylchloride. The antiseptic agent comprises a quaternary ammonium compound.

### CROSS REFERENCE TO RELATED APPLICATION

This application claims priority under 35 U.S.C. § 119(e) to Provisional Patent Application Ser. No. 60/724,175, filed on Oct. 6, 2005, the entire disclosure of which is hereby incorporated by reference.

### FIELD OF THE INVENTION

This invention relates to disposable gloves. In one aspect, the invention relates to plastic disposable gloves while in another aspect, the invention relates to plastic disposable gloves with their inner surface coated with an antiseptic agent. In yet another aspect, the invention relates to plastic disposable examination or surgical gloves treated with an antiseptic agent.

### BACKGROUND OF THE INVENTION

Plastic disposable gloves are used as a protective measure in a wide variety of applications. Not only do the gloves protect the hands of the wearer from the objects and surfaces contacted by the wearer, but the gloves also protect the objects and surfaces from the hands of the wearer. For example, mail handlers wear plastic gloves to protect themselves from any contamination on the mail, and food handlers wear plastic gloves to protect the food against contamination from their hands. Of course, medical, dental and other health care professionals have long worn plastic gloves during examination and surgical procedures to protect both themselves and their patients from the transfer of infectious disease.

Plastic gloves are typically made of thin, elastic material, e.g., natural or synthetic rubber latex, acrylonitrile, polyvinylchloride and the like, that minimize the space between the inside surface of the glove and the skin of the hand. This minimal space creates at least two difficulties in using these gloves, i.e., difficulty in donning or putting the glove onto the hand, and discomfort wearing the glove for an extended period of time, e.g., thirty or more minutes. Due to the nature of the material from which the glove is made, it does not slide easily onto the hand, particularly if the hand is wet. Due to poor air circulation within the glove once the glove is on the hand, the hand is prone to sweat. Prolonged wearing of these gloves results in a moist, warm environment within the glove (particularly if the hand was wet at the time the glove was donned) and this, in turn, allows for the growth of bacteria, yeast, fungus and the like. In turn, these conditions and the growth of these organisms can result in skin discomfort and possible infection.

In response to these problems, plastic gloves have undergone various modifications. For example, the inside of some gloves have been coated with a powder, e.g., talc, while others with a lotion, while still others with aloe vera. While all of these modifications are effective to one extent or another in easing the donning of the glove and wearing the glove over an extended period of time, all are subject to improvement. For example, sweat easily overcomes the effectiveness of the thin layer of powder on the inner surface of the gloves, and lotions and aloe vera can leave a greasy feeling on the hand. None, by themselves, offer antiseptic protection.

### SUMMARY OF THE INVENTION

In one embodiment of the invention, a disposable plastic glove is provided the inside surface of which carries an antiseptic agent. The glove is thin and elastic, and it can be made of any material that will produce a glove with such attributes. Preferred materials include both natural and synthetic rubber latex, acrylonitrile and polyvinylchloride. The antiseptic agent comprises a quaternary ammonium compound.

Any conventional process can make the plastic glove. Once made, the glove is inverted, if not already inverted, so that its interior surface is out and its exterior surface is in. The glove is then loaded into a dryer/tumbler, warmed to a predetermined temperature, and sprayed with the antiseptic agent. The sprayed glove is then dried, cooled and inverted back to its normal state, i.e., the interior surface in and the exterior surface out. The finished glove is easy to don over either a dry or wet hand, provides protection against bacteria, yeast, fungus and other infectious agents, and provides the skin of the hand with a cool, pleasant feeling over an extended period of time.

### DETAILED DESCRIPTION OF THE INVENTION

Any conventional process can make the basic glove of this invention, i.e., the glove prior to receiving a coating of the antiseptic agent on its interior surface. In one embodiment, the glove is made of natural or synthetic rubber latex while in other embodiments, the glove is made of acrylonitrile or polyvinylchloride. In a typical process, a form in the shape of a human hand is coated in any conventional manner, e.g., dipping, spraying, etc., dried, cleaned of residual material and contaminants, if any (e.g., excess latex, protein components within the latex, etc.), sterilized, optionally treated with an agent of one kind or another (e.g., silicone oil), dried and removed from the form, U.S. Pat. Nos. 6,274,154, 6,423,328 and 6,630,152, and the patents and references cited in each of these patents, provide a more complete description of this known process and these known materials of construction.

The glove can comprise a single layer or multiple layers, and the layers can be the same or different in composition and structure. The surfaces of the glove can be smooth or textured or a combination of the two. The glove can be of any size and shape, e.g., cuffed, extended for forearm protection (such as a surgical glove), pigmented and the like. The thickness of the glove may also vary to convenience.

In the manufacturing process, the surface of the glove formed next to the mold is typically the outer surface of the glove, and the surface of the glove open to the environment is the inner surface of the glove, i.e., the surface of the glove that will ultimately be in contact with the skin of the hand. In the process of stripping the glove from the mold, the glove is typically inverted to its intended, usable state, i.e., the interior surface of the glove is in (or on the inside the glove) and the exterior surface of the glove is out (or on the outside of the glove). After molding, the glove is often subjected to washing and drying.

At some point in the manufacturing process, particularly with respect to gloves made with natural or synthetic rubber latex, it is treated with a chlorine solution or chlorine gas. If the glove has not been treated with chlorine while still on the mold, then it is usually first applied to the outside surface of the glove, the glove then turned inside out, and then applied to the inner surface of the glove. Chlorine can help to sterilize the glove, wash off powders, and to dissolve residual proteins that could trigger allergic reactions among repeat users.

To efficiently apply the antiseptic agent to the glove, the glove should be inside out. If the glove is not already is such a state, e.g., from a chlorination treatment, then the glove is preferably placed into such a state before proceeding with the antiseptic treatment. The agent can be applied in any conventional manner, e.g., spraying, dipping and the like, but preferably it is applied in a tumble/spray process. This process employs a dryer/tumbler such as M San SS82-1 manufactured by San Motors of Samaedum, Bangkhuntirn, Bangkok, Thailand. Dry gloves in their inverted state are loaded into the dryer/tumbler, and tumbled while optionally raising the temperature within the machine. The amount of gloves (usually measured by weight) added to the machine is a function of the size of the machine, and the machine is usually loaded to maximize the efficiency of the process, i.e., it is loaded with as many inverted gloves as can be effectively treated with the antiseptic agent. The temperature within the machine is a function of a number of different variables, e.g., amount of gloves in the machine, the composition of the glove, whether not the glove has received a prior treatment, etc., but if the temperature within the tumbler/dryer is raised at all, then typically it is raised to between about 45 and about 60 C. The rate of rotation can vary widely, but typically it is between about 30 and about 35 revolutions per minute.

Once gloves have been loaded into the tumbler and the interior of the tumbler is at the desired temperature, then the tumbler will run without heat, or additional heat, and air circulation. At this moment the spray process begins. The antiseptic agent is sprayed onto the gloves while they continue to tumble. Any agent that imparts an antiseptic quality to the glove can be used in the practice of this invention, but a preferred agent is D DON-2™ available from Cosmic Discovery SDN.BHD of Selangor Darul Ehsan, Malaysia. This agent is a mixture of water, silicone oil, cetylpyridinium chloride (a quaternary ammonium salt) and other ingredients. The agent is diluted with water, and sprayed upon the tumbling gloves, typically intermittently and over a period of minutes. In one embodiment, 40 kilograms (kg) of gloves are tumbled at about 40-45 C., and over a period of about 45 minutes six liters of a 1.5 weight percent solution of D DON-2™ is sprayed onto the gloves. After the last of the antiseptic solution has been sprayed onto the gloves, the gloves continued to tumble until the agent is dried onto the gloves, the heat is removed and the gloves continue to tumble while the gloves cool, the tumbling stopped, the gloves removed from the machine, and the gloves inverted to their usable state, i.e., inside in and outside out. The gloves are then ready for quality control inspection and packaging. Enough antiseptic agent is sprayed onto each glove and covers enough inside surface of the glove to allow easy donning of the glove onto a wet or dry hand, preferably a wet hand. Preferably, the antiseptic agent completely and evenly covers the entire inside surface of the glove. In this manner, the therapeutic treatment to the hand offered by the antiseptic agent is maximized.

The following examples illustrate certain embodiments of the invention. Unless indicated to the contrary, all parts and percentages are by weight.

### EXAMPLES

Preparation of Spray Materials:

CTF:Polymer SSA/OLI-5 Blend

Two and one-half liters of CTF-3B (a viscous, milky white liquid comprising a polystearate compound and available from Cosmic Discovery SDN.BHD.) is poured into 200 liters of soft water and mixed well. After standing for 16 hours, 1.4 liters of Polymer SSA/OLI-5 (a milky white liquid comprising acrylic polymer and polydimethylsiloxane and also available from Cosmic Discovery SDN.BHD.) is poured into the diluted CTF and mixed well. After mixing the diluted CTF and SSA/OLI-5, 1 liter of Silcone DC-346 (a silicone emulsion available from Dow Corning Corporation) is added and the resulting blend is again mixed well. This final blend is applied to the gloves at a rate of 10 liters per 80 kilograms of gloves. This blend is a anti-tact agent for spraying onto the exterior surface of the glove. One hundred finished gloves are typically packed into a dispenser box, and this agent promotes non-adhesion among the gloves.

D DON-2

Six hundred and eighty milliliters of D DON-2 (available from Cosmic Discovery SDN.BHD of Selangor Darul Ehsan, Malaysia, and containing more than 20% cetylpyridinium chloride, less than 10% oil, and the remainder water and other ingredients) is poured into 6 liters of soft water and mixed well. The diluted D DON-2 is applied to the inside surface of the gloves at a rate of about 6 liters per about 40 kg of gloves.

### Example 1

Eighty kilograms of plastic examination gloves made from natural rubber latex are placed in a washing vat with 800 liters of water. The gloves are not inverted, i.e., the inside of the gloves are in and the outside out. The gloves are tumbled for 10 minutes, removed to another washing vat and washed under the same conditions for another 10 minutes. The wet gloves (20 kilograms) are then transferred to another machine in which the water is “extracted”, i.e., the gloves are tumbled at a high spin rate to remove as much water as possible. The gloves are typically spun for about 20 minutes.

Eighty kilograms of water-extracted gloves are then transferred to a drying tumbler (e.g. a San Motors model M San SS82-1), and tumbled for 10 minutes at 55-60 C., and then sprayed with 10 liters of the CTF:Polymer SSA/OLI-5 blend. The spraying and tumbling occur over a 60-90 minute period. The gloves are then allowed to cool to a temperature of 36-40 C. over a period of 10-20 minutes while the tumbling continues.

The gloves (80 kg) are then removed from the drying tumbler, turned inside out, and transferred to another drying tumbler. Here they are tumbled for another 60-90 minutes at 40-45 C. while sprayed with 12 liters of the diluted D DON-2 antiseptic agent. The gloves are allowed to cool to 36-40 C. while tumbling for an additional 10-20 minutes. The gloves are then removed from the drying tumbler, inverted back such that the exterior surface of the glove is out and the interior surface in, subjected to a quality control inspection, and packaged. The gloves are easy to don onto a wet hand, and are comfortable to wear for a period exceeding 30 minutes.

### Example 2

The procedure of Example 1 is repeated but in this instance, the gloves are not subjected to a separate wash and subsequent spray with the CTF:Polymer SSA/OLI-5 blend. Rather, the gloves are treated, after having been turned inside out, as received from the manufacturing line. Here again, the gloves are easy to don onto a wet hand, and are comfortable to wear for a period exceeding 30 minutes.

### Example 3

In this example, the gloves are first treated with chlorine gas, washed, rinsed, water-extracted, dried and weighed before being treated with the CTF:Polymer SSA/OLI-5 blend as in Examples 1 and 2. Once again, the gloves are easy to don onto a wet hand, and are comfortable to wear for a period exceeding 30 minutes.

Although the invention has been described in considerable detail, this detail is for the purpose of illustration. Many variations and modifications can be made on the invention without departing from its spirit and scope as described in the appended claims. All U.S. patents and allowed patent applications identified above are here incorporated by reference.

What is claimed is:

1. A plastic glove with an inside surface treated with an antiseptic agent.

2. The plastic glove of claim 1 in which the antiseptic agent comprises a quaternary ammonium compound.

3. The plastic glove of claim 2 in which the quaternary ammonium compound is cetylpyridinium chloride.

4. The plastic glove of claim 3 in which the plastic glove is made of at least one of natural or synthetic latex rubber, acrylonitrile and polyvinylchloride.

5. The plastic glove of claim 4 in which the plastic glove is an examination or surgical glove.

6. A method of treating a plastic glove with an antiseptic agent, the method comprising spraying an inside surface of the glove with an antiseptic agent while the glove is subjected to tumbling at an elevated temperature.

7. The method of claim 6 in which the antiseptic agent is sprayed onto the inside surface of the glove after the glove has been turned inside out, and while the glove is tumbled in a dryer at a temperature between 40-45 C.

8. The method of claim 7 in which the plastic glove is made of at least one of natural or synthetic latex rubber, acrylonitrile and polyvinylchloride and the antiseptic agent is a quaternary ammonium salt.

9. The method of claim 8 in which the antiseptic agent is at a concentration in water of about 1.5 weight percent.

10. The method of claim 9 in which the antiseptic agent is sprayed onto the inside surface of the glove at a rate of 6 liters of agent per 40 pounds of gloves.
